(19) United States
(12) Patent Application Publication
(10) Pub. No.: US 2025/0155331 A1
(43) Pub. Date: May 15, 2025

(54) System and Method for Pulsed Canister Sampling

(71) Applicant: Entech Instruments Inc., Simi Valley, CA (US)

(72) Inventor: Daniel B. CARDIN, Simi Valley, CA (US)

(21) Appl. No.: 18/946,778

(22) Filed: Nov. 13, 2024

Related U.S. Application Data
(60) Provisional application No. 63/598,385, filed on Nov. 13, 2023.

(51) Int. Cl. G01N 1/22; G01N 1/24; G01N 7/00; G01N 33/00

(52) U.S. Cl. CPC G01N 1/2273; G01N 1/24; G01N 7/00; G01N 33/0073

(57) ABSTRACT

A sampling system for collection of VOCs in air includes a canister and a valve configured to pulse open and closed for the constant introduction of an air sample into the canister over time. Throughout the duration of sampling, the time for which the valve is open for each pulse is modified to compensate for the increasing pressure in the canister, with temperature-compensated pressure readings obtained to ensure a constant mass flow of air into the receiving canister despite pressure changes due to temperature fluctuations during the sampling period.

### CROSS-REFERENCE TO RELATED APPLICATION

This application claims the benefit of U.S. Provisional Patent Application No. 63/598,385, filed on Nov. 13, 2023, the entire disclosure of which is incorporated herein by reference in its entirety for all purposes.

### FIELD OF THE DISCLOSURE

The disclosure relates to a system and method for collecting a gas phase sample in a canister using a pulsed valve.

### BACKGROUND OF THE DISCLOSURE

Both whole air canister sampling and sorbent tube sampling for either solvent extraction or thermal desorption have been used to collect chemicals in air for more than 40 years for subsequent analysis of the collected chemicals by GC or GCMS (Gas Chromatography/Mass Spectrometry). These devices can collect sample quickly by increasing the sampling flow rate, or over a longer period of time to perform time-weighted sampling. Very fast sampling can provide a “snapshot” measurement of the concentration of chemicals in the air, while time integrated sampling can be more informative by showing the average concentration of chemicals present over that sampling period, which in turn can be used to determine the total amount of the chemicals likely to enter into individuals exposed to these chemicals due mostly to inhalation and then partially to dermal absorption. Each chemical has a certain risk factor, so measuring the average concentration of each chemical through a speciated analysis will allow a better determination of overall risk when exposure to these chemicals occur.

Sampling onto sorbent tubes, rather than whole air sampling into vacuum canisters has a lot of limitations and challenges. With sorbent trapping, each sampling device is a part of the analytical sample preparation process because the sample is being changed/fractionated in the field by trying to retain some compounds while eliminating others (removal of water vapor, air/fixed gases, other unretained light compounds). It is very difficult to create sorbent traps that are perfectly identical, so the breakthrough volume and the recovery of many compounds tend to vary from tube to tube. In some cases where very light compounds must be measured, sorbent collection in the field simply cannot be used (Formaldehyde, C₂ Hydrocarbons, CF₄, C₂F₆, many other greenhouse gases). In addition, it would be prohibitively expensive to completely measure the recovery and background levels for all sorbent sampling tubes prior to analysis, so a lot of assumptions for consistency and accuracy are made. For vacuum canisters that are meant to be used up to hundreds of times, it is far more cost effective to confirm their ability to clean up to below required background levels, and to confirm they can recover all compounds of interest after a required holding period (1-4 weeks). Since canisters collect everything and are made out of impermeable materials such as stainless steel or glass, even Hydrogen, Methane, and the lightest greenhouse gases will be recovered. When taken to the laboratory, canisters are analyzed using sophisticated sample preconcentration systems, using cryogenic or electronic cold trapping, sophisticated moisture elimination methods, and then final focusing for rapid injection into a GCMS. Although sorbent traps can recover heavier compounds than are typically recovered using canisters, the ability to measure the complete light end of the spectrum out through about C12 Hydrocarbons is something that only vacuum sampling canisters are capable of.

Many flow controllers have been developed in the past 40 years to attempt to fill canisters at a constant fill rate over the required sampling period, but even the most accurate of these have several limitations. Canisters delivered to the field for sample collection typically have volumes between 1 L to 6 L, and even the larger 6 L canisters can be difficult to fill over a 24 hour period, as accurate flow rates in the 3.4 cc/min range are required. Calibration at this low level has proven difficult, as most flow controllers used in industry are not optimized at such a low flow rate, nor is the ability to verify flows at these levels. However, a 24-hour fill time is often not long enough to determine true risk assessment, as the average concentration in a particular area will change based on wind direction and wind speed. Given that meteorological conditions can change over several days to even weeks, a much longer sampling period is often needed to truly assess risk in a given area. Therefore, rather than a 24 hour time frame at 3.4 cc/min, as long as a 1 month period at flow rates down to 0.1 cc/min may be necessary to accurately determine the risk associated with working or living in a given area. Flow rates in the 0.1 to 1 cc/min have been virtually impossible to calibrate and maintain with previous canister sampling technology.

In addition to accurate calibration at very low flow rates, it is important that active flow rates are maintained above static diffusion rates, otherwise a positive bias can occur for lighter, faster molecules with lower cross-sectional areas that can diffuse further and faster, relative to heavier molecules with larger cross-sectional areas that diffuse through air more slowly. For example, Benzene can diffuse through a 5 mm wide by 15 mm long tube at 0.67 cc/min, while Xylenes can only diffuse through this zone at about 0.46 cc/min. Therefore, any sampling device operating at much slower active flow rates such as 0.1 to 0.2 cc/min (1 month or 2 week sampling into 6 L canisters, respectively) must arrest any diffuse component of sampling, otherwise a large bias will occur.

### SUMMARY OF THE DISCLOSURE

The disclosure relates to a system and method for collecting a sample in a canister using a pulsed valve. A completely new air sampling device for filling canisters at rates from 3 hours to 1 month is presented here that performs a “fill control” technique rather than a flow control, to precisely fill canisters at a constant rate. Temperature compensation of the pressure readings is performed to ensure that changes in temperature do not affect the determination and ability to control the rate of mass introduction into the canisters. To prevent any diffusive component from creating a bias in the results, two techniques are employed that work together. First, a very small orifice inlet is created, or an inert micron porous frit is used to virtually eliminate diffusive intake of chemicals. In addition, the new sampler “breaths the sample in”, to increase dynamic sampling component to again reduce diffusion induced bias. Feedback is provided by a 0-15 psia pressure sensor, with temperature compensation to ensure correct mass flow. The length of each breath is controlled to maintain a constant fill rate, both while the canister is at full vacuum, and when the canisters is getting close to atmospheric pressure.

Rates below a certain point in a traditional mass flow controller are not accurate because the diffusive, non-convective component to mass transfer becomes a larger faction to the total mass flow. Diffusion creates mass flow in all directions, creating erroneous and incorrect readings when using mass flow controllers. To eliminate this problem, the true volumetric sampling system described herein uses a restrictor placed just upstream of a fast-acting valve that can be pulsed on and off, or partially on (proportional valve) to control the flow of gas into a vacuum sampling vessel. A pressure sensor on that vessel-tracks the total amount of gas introduced overall and during time intervals as short as 0.001% of the sampling period. Measuring the incremental amount of gas introduced over a one month period, for example, with a 0.001% time interval accuracy means being able to measure and compensate for the pressure change in the canister about every 1 minute. Since pressure changes with temperature, an accurate temperature sensor tracks the temperature in the canister to compensate for any changing temperatures during sampling. This is not the temperature compensation that is used to make sure that piezo electric pressure sensors and associated electronics produce readings that are not affected by temperature, but rather the compensation for the expansion and contraction of the collected gas in the canister, which in the past has never been considered to determine the true mass of gas sampled into a canister. Sampling can be started via an external start switch, via a remote connection using Blue Tooth, WiFi, or Cellular communications, or by setting a start date and time, and a sampling duration as monitored by a real time clock as a part of the sampler control electronics. This sampling device based on collected pressure inside of the vacuum canister allows far easier calibration than prior mechanical back-pressure regulators, making sampling much more reliable, either for short term or long term sampling. This approach can bring accurate sample collection into the hands of non-trained citizens who are concerned about long term risks associated with breathing air in their homes or in the communities where they live or work.

### BRIEF DESCRIPTION OF THE DRAWINGS

FIG. 1 illustrates a sampling system according to some examples of the disclosure.

FIG. 2 illustrates additional components of the sampling system according to some examples of the disclosure.

FIG. 3 illustrates a plot of the temperature-compensated pressure over time and a plot of the on time for the control valve over time during sample collection using a system according to some embodiments of the disclosure.

FIG. 4 illustrates a method of collecting a gas sample using techniques according to some embodiments of the disclosure.

### DETAILED DESCRIPTION

In the following description, reference is made to the accompanying drawings which form a part hereof, and in which it is shown by way of illustration specific examples that can be practiced. It is to be understood that other examples can be used, and structural changes can be made without departing from the scope of the examples of the disclosure.

The disclosure relates to a system and method for collecting a sample in a canister using a pulsed valve. A completely new air sampling device for filling canisters at rates from 3 hours to 1 month is presented here that performs a “fill control” technique rather than a flow control, to precisely fill canisters at a constant rate. Temperature compensation of the pressure readings is performed to ensure that changes in temperature do not affect the determination and ability to control the rate of mass introduction into the canisters. To prevent any diffusive component from creating a bias in the results, two techniques are employed that work together. First, a very small orifice inlet is created, or an inert micron porous frit is used to virtually eliminate diffusive intake of chemicals. In addition, the new sampler “breaths the sample in”, to increase dynamic sampling component to again reduce diffusion induced bias. Feedback is provided by a 0-15 psia pressure sensor, with temperature compensation to ensure correct mass flow. The length of each breath is controlled to maintain a constant fill rate, both while the canister is at full vacuum, and when the canisters is getting close to atmospheric pressure.

Rates below a certain point in a traditional mass flow controller are not accurate because the diffusive, non-convective component to mass transfer becomes a larger faction to the total mass flow. Diffusion creates mass flow in all directions, creating erroneous and incorrect readings when using mass flow controllers. To eliminate this problem, the true volumetric sampling system described herein uses a restrictor placed just upstream of a fast-acting valve that can be pulsed on and off, or partially on (proportional valve) to control the flow of gas into a vacuum sampling vessel. A pressure sensor on that vessel-tracks the total amount of gas introduced overall and during time intervals as short as 0.001% of the sampling period. Measuring the incremental amount of gas introduced over a one month period, for example, with a 0.001% time interval accuracy means being able to measure and compensate for the pressure change in the canister about every 1 minute. Since pressure changes with temperature, an accurate temperature sensor tracks the temperature in the canister to compensate for any changing temperatures during sampling. This is not the temperature compensation that is used to make sure that piezo electric pressure sensors and associated electronics produce readings that are not affected by temperature, but rather the compensation for the expansion and contraction of the collected gas in the canister, which in the past has never been considered to determine the true mass of gas sampled into a canister. Sampling can be started via an external start switch, via a remote connection using Blue Tooth, WiFi, or Cellular communications, or by setting a start date and time, and a sampling duration as monitored by a real time clock as a part of the sampler control electronics. This sampling device based on collected pressure inside of the vacuum canister allows far easier calibration than prior mechanical back-pressure regulators, making sampling much more reliable, either for short term or long term sampling. This approach can bring accurate sample collection into the hands of non-trained citizens who are concerned about long term risks associated with breathing air in their homes or in the communities where they live or work.

Other systems and methods for filling canisters over longer periods of time have been problematic, so the longest time integrated sampling stated in any US EPA Method is just 24 hours. Such a short time integrated sampling period will not typically provide the best determination of risk when measuring outside air, as wind directions can change over a period of time. A time collection of 1-4 weeks would provide a much more accurate determination of the average exposure for occupants who spend time in those areas. For example, toxicologists have determined that an elevated three-week exposure to Trichloroethylene (TCE) in the first trimester of pregnancy can increase the risk of heart defects in the developing fetus, so longer sampling times that cover this full period of time is very important. Mechanical and even mass flow-controlled sampling devices in use today are not able to control the flow rates into 1-6 L canisters over such a long period of time, because the flow rates are simply too slow for these devices to control properly. Filling a 1.4 L canister over a two-week period for example would require sampling rates of just 0.07 cc/min which is nearly 10× below the diffusion rate of many VOCs into a 5 mm opening at the end of a sorbent trap, even when no pump is actually moving air, so special considerations would be needed to allow sampling at such low flow rates while accurately measuring compound concentrations in air.

In addition, any “open loop” sampler that did not confirm the accurate filling of the sampling canister on a continuous basis would too often fail to obtain a reliable sample. When canisters are retrieved after sampling, it is important that there be at least a small amount of vacuum left in the canister to confirm that sampling was still occurring at a constant rate right up to the end of the sampling event. On the other hand, if too much vacuum is left in the canister, there may not be enough sample in the canister to allow the analyzer to reach required detection limits. Most sample preconcentration systems cannot draw a vacuum canister to much under 0.5 atmospheres (7.4 psia or 50.7 kPa) without starting to create errors in the analysis. Hence, it's extremely important to fill a canister close to atmospheric pressure, but not all the way to atmospheric pressure. This narrow range of acceptable final pressures in canisters has required a lot of expertise in the past to collect vacuum canister samples properly, adding significantly to the cost of performing this kind of air monitoring, especially if canister sampling has to be repeated due to not finishing within the limited, final target vacuum range. Systems and methods according to the disclosure address the shortcomings of other sample collection systems and methods using canisters.

A sampling system and method according to the disclosure simplifies the collection of whole air samples using vacuum canisters to measure volatile chemicals in air at concentrations in the range of low part per trillion to percent levels. The sampling system includes a canister and a sampling device coupled to the canister inlet that includes a filtering restrictor and a valve configured to pulse on and off. The system further includes an absolute pressure sensor coupled to an A/D converter and one or more temperature sensors positioned on or in the canister and/or proximate to the sampling device.

Sampling begins based on a scheduled start date and time, or in response to detecting a trigger, such as activation of a button or switch. The system measures the local atmospheric pressure and the current pressure in the canister and determines, based on a target ending pressure and sampling duration, a fill rate for the canister. The sampling device pulses the valve on and off at regular intervals throughout the sampling process and monitors the pressure and temperature of the canister. Software calculates an appropriate duration to open the valve for each pulse to collect the sample at a consistent, time-weighted average mass flow rate over time. Changes in temperature inside the canister (e.g., due to weather and time of day) and/or pressure inside the canister (e.g., due to gradually collecting sample and, to a lesser extent, changes in temperature) necessitate adjusting the duration for which the valve is opened during sampling pulses to maintain a substantially constant rate of sampling when expressed in terms of mass. During sampling, the system monitors the pressure in the canister using the absolute pressure sensor and A/D converter.

While collecting sample, the system targets a final pressure in the canister that is a predetermined pressure level below atmospheric pressure at the location of the sample at the time of sampling. The system uses the pressure sensor to target the desired final pressure in the canister. It is desirable for the final pressure in the canister to be as close to the target final pressure as possible without being greater than the target final pressure. EPA air analysis methods require the final pressure in the canister to be either 2″ Hg (6.77 kPA) or 3″ Hg (10.16 kPa) (method dependent) under local atmospheric pressure, and no higher. A pressure level in the canister equal to local atmospheric pressure would stop the introduction of air, so keeping the final pressure in the canister below local atmospheric pressure indicates that sample was being collected through the end of the sampling period. Coming very close to the allowable maximum collected pressure in the canister provides more sample for analysis, helps to cover up any active sites in the canister without affecting concentrations as much, and finally can further dilute any remaining contaminants in the canister left over from the previous sampling event. Thus, it is desirable for the final pressure in the canister to be as close to the target final pressure as possible without exceeding the target final pressure.

Pressure within a closed system, such as the sample canister while the intake valve is closed, fluctuates due to fluctuations in temperature. The temperature in the canister fluctuates during the sample collection process due to changing temperatures throughout the day (e.g., being colder at night and warmer during the day—and due to presence or absence of direct Sun exposure. The system uses a temperature sensor to monitor the temperature in the canister and/or at the system, so that one or more processors of the system are able to calculate a temperature compensated pressure reading. The system uses the temperature compensated pressure reading to monitor the mass of the sample collected in the canister and to ensure a constant rate of sample mass collection with each opening pulse of the intake valve so a true mass introduced per minute can be determined to ensure constant mass flow whether the temperature is high or low, increasing or decreasing. It is possible to set the temperature compensation to a specific temperature, such as 25 degrees Celsius, which is close to the temperature of air labs, such that the true pressure in the canister will be near the 25 degrees Celsius pressure compensated temperature, making it easy to confirm no leaks into the canister occurred simply by using an absolute pressure sensor to measure the pressure of the canister with no additional temperature compensation needed.

During filling, an absolute temperature compensated pressure difference for each on/off pulse is targeted based on the fill time and the size of the canister. Since the canister starts off at full vacuum, there is virtually no effect on pressure based on the current temperature. That allows a valve pulse on time to be established with no temperature effects on pressure based on a less than perfect determination of the current, average temperature of the vacuum container. During sampling, the pulse on time slowly increases in a very predictable way, so the control algorithm can use this to ensure that sudden changes in temperature do not affect the mass collection rate by a significant amount. The temperature of the canister is monitored in a way that closely represents the average temperature, with the part of the canister monitored shielded from direct sunlight or direct air exposure. However, sunlight hitting the canister unevenly can produce an uneven temperature, and the control algorithm does not over-react based on a sudden rise in pressure. This temperature inconsistency is typically short lived as the canister thermalizes, so a change of no more than 10% of the recent pulse on times are made in response to fast changing pressures, which allows the mass flow rates to stay within even the most strict sampling guidelines from both the US EPA and OSHA. The internal control electronics stores the pressure and temperature compensated pressures for later graphical representation to show the ongoing rate of mass introduction into the canister, and a straight line increase during the sampling period is the goal for temperature compensated pressure rise

Systems and methods according to the disclosure can be used for very small systems such as personal monitors that are worn near the breathing zone, and to fill larger canisters for workplace area monitoring. High or low fill rates can be selected to fill large canisters for ambient air or low-level indoor air analysis, or to fill small to medium size canisters for factory or fenceline monitoring, for stack gas emission monitoring, for automobile or airliner cabin air monitoring, for schools, hospitals, or office and government buildings. This device can be used to collect both organic and PFAS (PerFluorinated Alkyl Substances) compounds, to determine risk levels for individuals living, working, or otherwise occupying these areas.

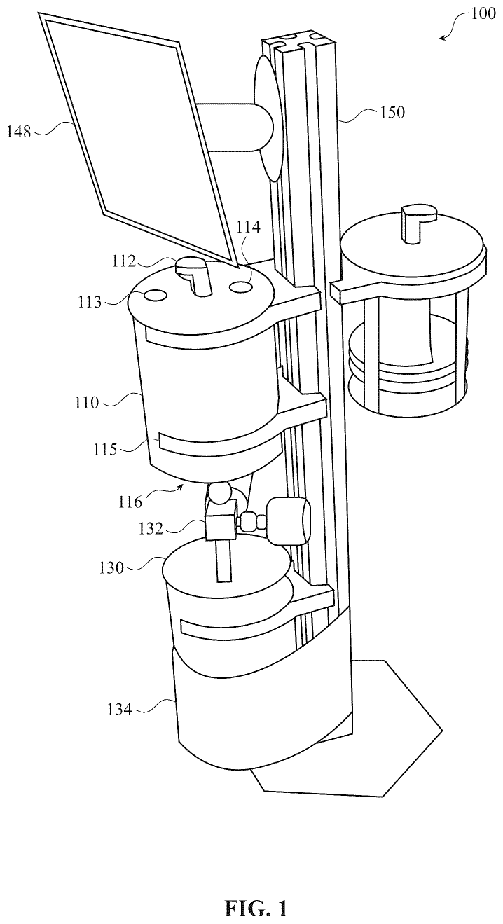

FIG. 1 illustrates a sampling system 100 according to some examples of the disclosure. Sampling system 100 includes a canister 130, insulator 134, isolation valve 132, connector 116, enclosure 110, brackets 115, connector 114, inlet 112, optional solar panel 148, and mount 150 described in further detail below.

Canister 130 is initially evacuated prior to the beginning of sample collection to provide a clean container for the sample free of any contaminants and to generate negative pressure relative to the environment, thereby facilitating flow of gas into canister 130 during sampling pulses described herein. Canister 130 is shown attached to the bottom of enclosure 110, using an isolation valve 132. Isolation valve 132 can also be used to seal the canister 130 before and after sampling to avoid contamination. A weather tight connector 116 on the bottom of enclosure 110 allows the temperature of the air being sampled to be accessed using a small thermocouple, or an extended thermocouple wire can be used to determine the average temperature below a thermal insulator 134 that allows the true, average temperature of the canister 130 to be determined.

Thermal insulator 134 optionally includes an insulating blanket or another insulating material formed or attached around the canister 130. The use of thermal insulator 134 causes the transfer of heat along the walls of the canister to be faster than the transfer of heat directly to the canister 130 from sunlight or from outside air. In this way, a representative temperature of the canister 130 can be determined. This thermal insulator 134 can cover some, most, or all of the canister 130 as needed to ensure a representative temperature is being obtained. Diurnal temperature fluctuations especially when sampling outside air can be significant, and direct sun exposure without insulation can also heat up the canister 130 to create a higher pressure in the canister 130 than pressures present at lower temperatures. These temperature fluctuations can interfere with measurements of how much sample has already been collected, which is determined based on measuring the pressure within canister 130. For example, without reducing temperature variation and/or compensating for temperature variation as described herein, an increase in canister 130 temperature could cause an increase in canister 130 pressure. In turn, in this example, the sampler may decrease the rate of sample introduction because the observed increase in pressure may assume to be caused by an increase in sample mass previously collected. However, by compensating for this higher or lower temperature, the system can determine what the standard pressure at 25° C. would be, so that a constant rate of increase in air mass inside of the canister 130 can be achieved. In some embodiments, such as when sampling indoor air, insulator 134 can be omitted.

Canister sampling systems are often placed inside of metal cabinets for purpose of protection from theft and for further protection from the elements. The sample inlet can either be inside of the cabinet if ample vents are present in the cabinet to ensure representative air is presented to the sampler, or inlet tubing can be introduced through the top of the cabinet to bring fresh air in from outside, and this may be preferred if any materials of construction of the cabinet may interfere with proper sampling, such as either absorption of chemicals in the air, or outgassing from something inside of the cabinet. When a cabinet is used, a temperature sensor at the bottom of the sampler near 116 can be used to measure the temperature in the cabinet to provide temperature compensated pressure readings. Inside of a cabinet, temperature differences between the bottom of the sampler and the canister are not expected, due to the inability to have inconsistent Sun exposure on the canister relative to the sampler.

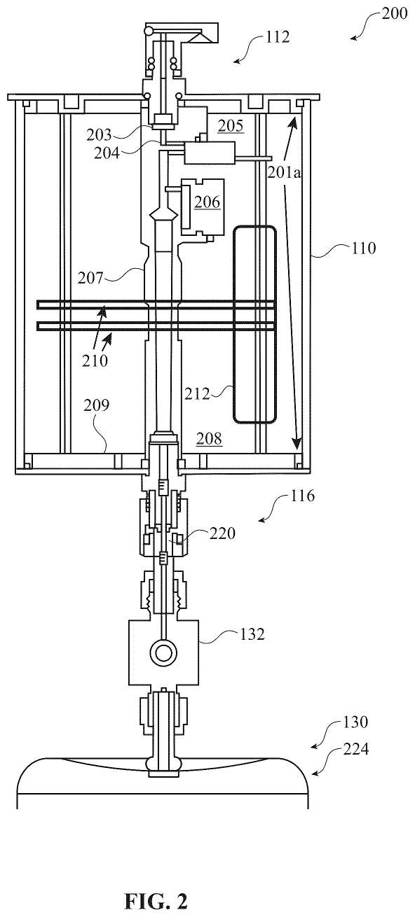

A weather-tight enclosure 110 houses the combined valve control, pressure sensing, temperature sensing device that determines the current temperature of the canister 130 when the sampler is inside of a protective cabinet. An external sensor is used to measure the temperature of the canister when no protective cabinet is used. Enclosure 110 further includes weather tight IO connections. The enclosure 110 can be supported by the canister 130 for indoor air sampling, or in a more secure way by attaching it to a mount 150 in the field, either using brackets 115, or by creating a rail securing tab as a part of the enclosure 110 for direct rail attachment. Additional components housed within enclosure 110 are described below with reference to FIG. 2.

An inlet 112 with a very small opening facing down provides ample exposure to the surrounding air mass, while substantially eliminating the preferential collection of faster moving, lighter molecules relative to slower, heavier molecules. Alternatively, a ceramic coated stainless steel frit can be placed at the inlet to further eliminate the diffusion of gases into the inlet between valve sampling events. Therefore, “active sampling” when the valve is open will equally represent both smaller, faster diffusing molecules, and heavier, slower diffusing molecules Inlet 112 is fluidly coupled to the components within enclosure 110, as shown in more detail in FIG. 2. A weather tight connector 114 allows access to a power source either for charging an internal battery, or for long term sample collection using a solar panel 148, or some other power source. One possible construction of the sampling system 100 shows that internal components (PCBs, batteries, flow mechanism) can be housed inside of this enclosure for field deployment. An LED feedback 113 on top of the controller uses different colored LEDs and blinking patterns to provide a wide array of feedback during field sampling.

FIG. 2 illustrates additional components of the sampling system 100 according to some examples of the disclosure. In particular, FIG. 2 includes a cutaway view of the inside of enclosure 110 and the components within the enclosure 110. Since this device contains electronics and other components that may be sensitive to excess water exposure, the enclosure 110 is sealed at the top and bottom using O-rings 201a. Any other access to the outside such as the power connection or thermocouple connection is also implemented using a means to prevent the introduction of moisture. A silica gel pack can be placed inside of the enclosure to absorb any water that diffuses through the O-rings 201a and/or other openings of enclosure 110, and eventually the enclosure 110 can be opened up to replace the silica gel desiccant.

Inlet 112 has been designed to prevent rain introduction, with an inverted cone to prevent exposure of liquid water at the inlet. A small amount of liquid water at the inlet 112 will not adversely affect the operation, as the vacuum applied will be sufficient to keep this path open for air, even when drawing in some moisture, as IDs of 0.005-0.02″ (0.01 cm to 0.0508 cm) are large enough so that water cannot typically clog them when vacuum is created by pulsing the sample control valve on and off. This inlet 112 is an advantage over many mechanical flow controllers in use today that use a critical orifice just prior to the mechanical back pressure regular where openings as small as 0.001″ (0.00254 cm) are used, as these extremely small openings are subject to clogging with even milligrams of condensed water. The inlet 112 is made out of 316 stainless steel or other high quality stainless steel, and can be coated with a ceramic known to be inert towards chemicals to be sampled. Inlet 112 can also be extended further above the top of enclosure 110, to avoid splash introduction of water during strong rain events, or to extend the inlet height to access outside air when using a cabinet enclosure.

Likewise, the additional portions (e.g., an entirety) of the flow path, including inlet flow path 204 and/or outlet flow path 207, through the sampling system 100 can be coated with a similar, inert ceramic, such as one deposited by CVD-Chemical Vapor Deposition. Downstream of the inlet 112 is an inlet restrictor 203 including a frit and/or filter that prevents any particles from reaching the control valve 205, and provides the necessary restriction to allow the proper air mass to be introduced. The volume of inlet flow path 204 is kept very small so that any pulsing of the valve 205 will draw very little unrestricted air through the valve 205 and into the canister 130. That is, with a controlled on time of just 10 msec, any volume downstream of the inlet restrictor 203 will be drawn almost instantly into the canister 130, not allowing a pulsed sampling approach to control the volume sampled very well if the volume of inlet flow path 204 was too large. At a flow rate of 0.07 cc/min, or 70 ul/min, an inlet flow path 204 having a volume of just 10-20 ul will allow a control of 70 ul/min by turning on the valve 205 for 10 ms plus some additional time to introduce a total volume of 70 ul through the valve 205. After this period, the valve 205 is closed until the next sip of air is introduced, which may be 15 seconds, 30 seconds, 1 minute, or another period of time later. This is similar to how a breath is taken, as even this is a pulsed process, and not continuous. In general, an air mass whether indoors or outdoors will generally not change faster than every few minutes, so sampling at a frequency of once per minute is considered to be a continuous sampling process. However, this is not a limitation of this sampling device, as even a lower volume of inlet flow path 204 can be used to “breathe” sample into the canister as frequently as people inhale a breath, or 6-10 times per minute.

Outlet flow path 207 is downstream of the control valve 205. The volume of the outlet flow path 207 is not as critical as the volume of the inlet flow path 204. However, the volume is still intended to remain reasonably small, as the initial connection of the enclosure 110 to the canister 130 will draw the gas contained in the exit flow path 204 into the canister 130. Valve connection 220 uses quick connect valves allowing the volume in the outlet flow path 207 to be isolated during transportation from the laboratory to the sampling location, using Ultra High Purity (UHP) Nitrogen or some other gas that will not contaminate the canister upon initial connection. Otherwise, for a continuous sampling application where the sampling system 100 is left at the sampling location and only the canisters 130 are swapped out to start the next collection, the air in outlet flow path 207 will have been recently introduced from the current sampling location so will be considered a representative aliquot of that air, so will not create a contamination problem. Canisters 224 often have a secondary valve 222 that is used as additional leak prevention.

Pressure sensor 206 is an absolute sensor reading from 0-15 psi (0-104 kPa) absolute, so will be accurate at any elevation in which the sampling is to occur. Pressure sensor 206 is temperature-compensated to read accurately at any temperature between about-30C to 100C, but as explained earlier, this is secondary to the completely different temperature compensation that corrects for the expansion of gas collected in the canister. Pressure sensor 206 is fluidly coupled to the inside of canister 130 through the outlet flow path 207, valve connection 220, and secondary valve 222.

Electronic Printed Control Boards (PCBs) 210 including one or more processors are located in the enclosure 110 to provide control of valve 205, the determination of the canister 130 pressure using sensor 206, and the reading of the thermocouple or other temperature sensing device that is placed on the surface of the canister 130 to compensate for the expansion and contraction of gasses in the canister 130 due to changes in temperature. For example, the thermocouple can be placed between the outer surface of the canister 130 and the insulator 134 in FIG. 1 and/or at the location 209 within enclosure 110 in FIG. 2. Battery 212 is used to power the electronics on the PCBs 210, the control valve 205, and the pressure sensor 206. Battery 212 can have enough power to allow sampling for several days and potentially for a few weeks, but for continuous applications where the sampler will be left in the field indefinitely, connector 114 allows a solar panel 148 or other power source to maintain power levels in the battery 212. A solar panel can be used that supplies enough power so that even during extended periods of cloudy weather during very short Winter daylight hours, enough power is generated to continue operating for months while performing continuous sampling where new vacuum canisters are swapped out regularly to obtain a true average measurement of air quality over very long periods of time.

For discrete sampling events such as monitoring the indoor or outdoor air quality at a location just one time, and for a duration of 2 weeks or less, the sampling system 100 may be brought to the field without a power source in addition to battery 212. The circuits on PCBs 210 can have Bluetooth or WiFi connection to the onboard processor(s) to allow external communication without making a physical connection. In addition, a weather tight connection on the bottom of enclosure 110 can be used to connect to an external device to allow programming or commencement of the sampling event remotely. This same panel on the bottom of enclosure 110 can have a button and/or switch that can either start a new sampling event by pressing if for a period of time, or can wake the sampler up from a low power sleep mode by momentarily depressing this switch, which allows the system to reduce power consumption during period of inactivity.

The sampling system 100 is programmed to indicate a sampling time in hours or days (0.25 hours to 30 days). As long as canisters from 1 L to 6 L are used, the sampling system 100 can be placed in a mode such that it will assume a 3 L volume, but will be able to tell within 0.01% of the sampling period which size canister is actually attached. That is, even for a two-week sampling event, the sampling system 100 will determine within 2 minutes which size canister is being used, and will adjust the sample introduction pulses accordingly. This adjustment occurs while the vacuum inside of the canister is still very strong, so almost no temperature errors can occur to create inaccurate readings even when the canister+sampler are unevenly exposed to strong, direct sunlight.—This can include adjustments to the durations for which the valve 205 is open for each pulse and/or the frequency of the pulses. This adaptability is an advantage over other canister sampling devices that have a limited range of sampling times (factor of 3 for many mechanical controllers, and a factor of 10 for mass flow controllers). Selecting sampling times from as little as 0.25 hours to as long as 30 days without changing anything in sampling system 100 provides a factor of nearly 3000×, from the shortest to the longest duration, without changing the inlet restrictor 203. However, by changing this restrictor 203 to a less restrictive restrictor, a grab sample can be taken within a few minutes to “capture” an event as necessary. That is, when placed at a sampling location where a trigger pulse or contact closer is created to indicate the need to take a quick snapshot of the air quality at that moment, sampling system 100 can be configured to do that as well.

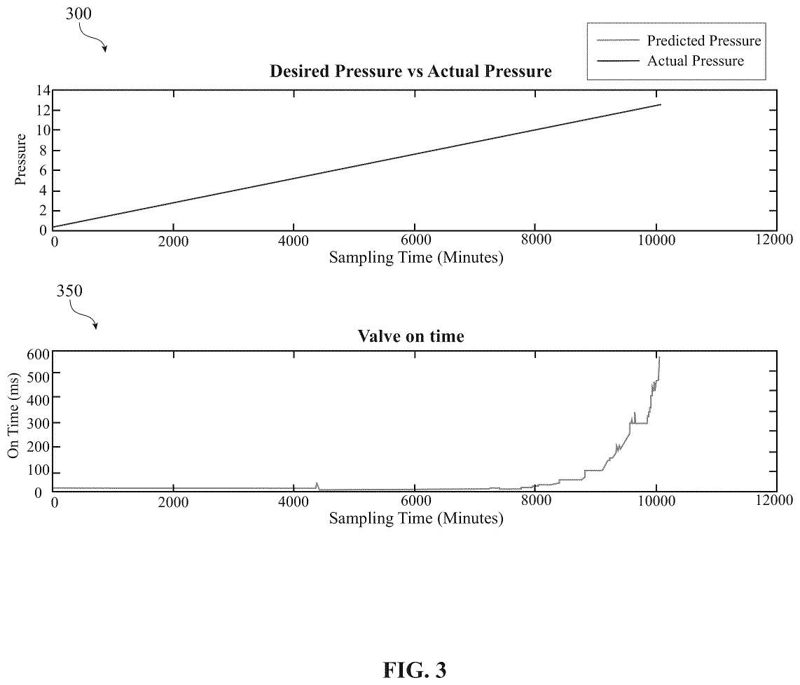

FIG. 3 illustrates a plot 300 of the temperature-compensated pressure over time and a plot 350 of the on time for the control valve over time during sample collection using a system according to some embodiments of the disclosure. Plots 300 and 350 include data collected while filling a 1.4 L canister over a seven-day period. This data can be used to verify that sampling occurred correctly.

The valve on time, illustrated in plot 350, must increase as the canister 130 fills, as the pressure differential between the canister and the outside air decreases, otherwise the rate of mass transfer into the canister 130 would decrease as well. Note that in any given location on the curve of plot 350, the on time does not change more than 5% to avoid creating a positive or negative bias in response to some external factors such as momentary introduction of moisture in the inlet or an inaccurate reading of the average canister temperature. In this way the mass flow into the canister remains virtually constant. In this example, the atmospheric pressure at the sampling location was 13.3 psia (91.7 kPa) at an elevation of 2400 feet (731.5 meters), so a target final pressure of 12.3 psia (84.8 kPa) is selected. The final pressure obtained after sampling was 12.3 psia (84.8 kPa), so less than a 0.1 psi (0.7 kPa) error was obtained relative to the requested final target pressure.

Achieving a final pressure that is substantially the same as the target pressure is important to ensure that sampling occurs right to the very end of the sampling period while filling the canister 130 with as much sample as possible. Many air sampling professionals currently choose to use lower sampling rates to ensure they are well under the maximum allowed fill pressure to improve their odds that the sample will be within the allowable range set by many State and Federal sampling methods. But underfilling the canisters in this way is at the expense of requiring larger canisters to be used in order to recover enough sample in the lab to meet method detection limits and/or to potentially run the canister samples more than once if needed. This practice increases the cost of performing this analysis, as larger canisters cost more than smaller canisters, are more expensive to ship, and take up more space in the laboratory. Larger canisters also cannot be analyzed on today's high efficiency robotic autosamplers that provide higher quality data by using shorter connections to the analyzer, and by making all samples flow through the same analyzer inlet, vastly improving the reliability of the analysis compared to multiple line rotary valve autosamplers that can have different levels of contamination in each inlet line. Hence, it is important to be able to use smaller, robotic lab autosampler-compatible canisters, by being able to fill these canisters reliably to near atmospheric pressure at a constant rate every time, even when the person operating the equipment is not highly qualified.

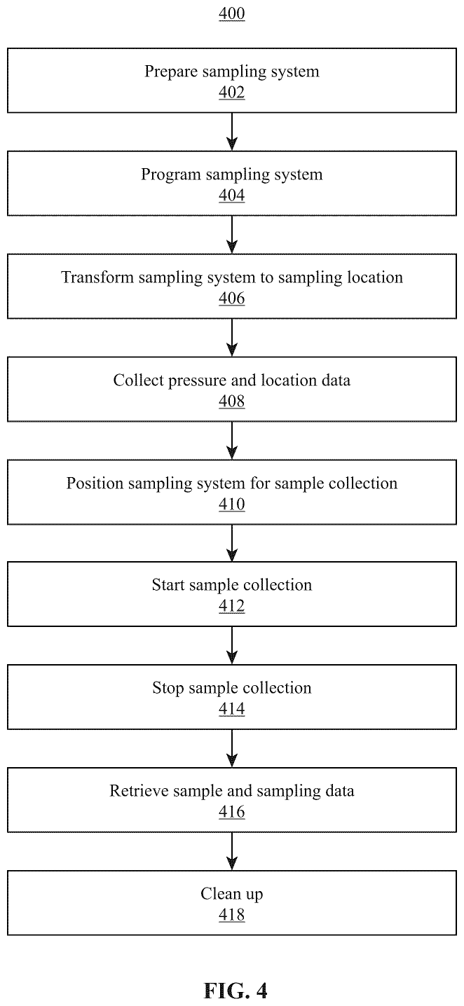

FIG. 4 illustrates a method 400 of collecting a gas sample using techniques according to some embodiments of the disclosure. Method 400 can be performed using sampling system 100 described herein. In some embodiments, method 400 is controlled at least in part with one or more processors in communication with the sampling system 100 via wired or wireless connection(s). Steps in method 400 can be repeated, performing in an order other than the order described, and/or omitted without departing from the scope of the disclosure.

At 402, the sampling system 100 is prepared. Preparation optionally includes charging any batteries incorporated into the sampling system 100 and/or flushing the inlet 112 and/or outlet 207 of the sampling system 100 with UHP Nitrogen gas and/or Zero air to remove contamination from the sampling system 100 prior to use. Occasional calibration of the internal pressure sensor using a vacuum pump and local atmospheric pressure can be performed regularly either by manual or automated valves that repeatedly vent and evacuate the outlet of the sampler during calibration.

At 404, the sampling system 100 is programmed. Programming the sampling system 100 optionally includes selecting a start time, sampling duration, and/or a final pressure relative to local atmospheric pressure (typically 1 psi, 2″ Hg, or 6.7-6.8 kPa) to be achieved. If the sampling will be started using the start button on the bottom of the sampling system 100, then no start time needs to be programmed, only the duration and target pressure under local atmosphere.

At 406, the sampling system 100 is transported to the location at which the sample is to be collected. Indoor or outdoor sampling locations are possible.

At 408, the sampling system 100 collects pressure and/or location information at the sampling location. This operation includes activating Bluetooth and/or GPS components of the sampling system 100. Additionally, the sampling system 100 measures and records the local atmospheric pressure prior to connecting the canister to the connector 116. In some embodiments, the sampling system 100 collects the pressure sample in response to detecting a button press. In response to detecting activation of the momentary switch at the bottom of the sampling system 100, the sampling system 100 wakes up, records local atmospheric pressure, and activates GPS to record the sampling site location.

At 410, the sampling system 100 is positioned for sample collection. For outdoor sample collection, the sampling system 100 can be placed on a rail support, in a cabinet, or held roughly two meters above ground level using some other support. Additionally, if sampling outdoors where sunlight can reach the canister 130, the canister 130 can be insulated by wrapping the canister 130 in insulation and a thermocouple can be placed near the center of the canister 130. For indoor sample collection, the sampling system 100 can be placed on the canister 130 sitting on a tabletop, shelf, or other surface. For indoor sampling, no external thermocouple is needed, as the temperature sensor at the bottom of enclosure 110 will be the same as the temperature inside the canister 130.

At 412 the sampling system 100 starts sampling. In some embodiments, the sampling system 100 starts sampling in response to detecting the momentary switch on the bottom of the sampling system 100 being activated. For example, the switch is activated in response to being pressed for a predetermined non-zero amount of time, such as 10 seconds or several times in succession, such as seven times or more. An indicator of the sampling system 100 will signify that sampling has started, such as illuminating an LED at the top of the sampling system 100. In some embodiments, the sampling system 100 starts sampling based on a predetermined schedule. In these embodiments, when a clock of the sampling system 100 indicates it is the predetermined time to start sampling, the sampling system 100 begins sampling. In some embodiments, the sampling system 100 starts sampling in response to receiving a signal from a remote device. In these embodiments, a communication device (e.g., a cellular module, a local analyzer with a trigger pulse, etc.) can be attached to the sampling system 100 via an external cable. A cellular module can communicate with another device via a wireless network to receive a signal to begin sampling. A local analyzer with a trigger pulse can provide the signal to begin sampling via a wired or wireless connection.

As described above, during the sampling process, the sampling system 100 opens the valve 205 at predetermined intervals for durations of time that vary throughout the sampling process. Varying the on time of the valve 205 maintains a substantially constant rate of mass transfer of sample into the canister 130. Throughout sampling, the system 100 monitors the pressure in the canister 130 using pressure sensor 206 and compensates the pressure for fluctuations in temperature in the canister 130 as described herein. The system 100 uses an algorithm stored in memory and executed with one or more processors to control the mass flow rate based on elapsed time and pressure inside the canister 130. In some embodiments, the algorithm targets filling the canister by an amount proportionate to the amount of the sampling duration that has passed, such as targeting filling the canister 5% of the way between the starting vacuum and the final target pressure at the time 5% of the sampling duration has passed; filling the canister 20% of the way between the starting vacuum and the final target pressure at the time 20% of the sampling duration has passed and so on. It should be noted, however, that sampling terminates before the canister 130 is 100% full to verify that sample was being collected up until termination of the sampling process as described herein. The target final pressure should be slightly less than the maximum allowable pressure relative to local atmospheric pressure as set by agency methods, for example the setpoint below local pressure may be 2.5″ Hg (8.46 kPa) if agency methods require at least 2″ Hg (6.77 kPa) vacuum remaining in the canister

At 414, the sampling system 100 stops collecting sample in response to the canister 130 reaching the target pressure and/or in response to a predetermined sample duration or stop time being reached. The canister will remain isolated (e.g., closed) until retrieved.

At 416, the sample and sampling data are retrieved for analysis. Retrieving the sample includes disconnecting the canister 130 from the sampling system 100. The canister 130 can be transported to a lab for analysis of the sample contained in the canister. Sampling data, such as pressure data illustrated by plot 300 and/or pulse time illustrated by plot 350 can be saved from memory of the sampling system 100 to another device for review and/or analysis, including verification that sample collection occurred as planned for the duration of sample collection. In some embodiments, the sampling system 100 transmits this data via Bluetooth to another device, such as a mobile device (e.g., a table or smart phone) or a computer (e.g., a laptop or a desktop computer).

At 418, the sampling system 100 is cleaned up for re-use. For example, cleanup includes one or more steps described above with respect to preparing the sampling system 100 at 402, such as flushing the sampling system 100 and charging its battery/ies.

Several operations of method 400 occur in response to detecting interaction with a button or switch integrated with the sampling system 100. These operations include:
1. In response to detecting one press, wake up sampling system 100 from a sleep state and a first visual indication is presented, such as a blue LED flash.
2. In response to detecting two button presses while the system is awake, record local atmospheric pressure. In response to a pressure error, such as detecting a pressure below a threshold (e.g., 7 psia or 48 kPa), the system presents a second visual indication, such as two amber LED flashes. In response to successfully recording the local atmospheric pressure, the system presents a third visual indication, such as two blue LED flashes.
3. In response to detecting three button presses, the system performs leak and flow test, and assumes the user has attached the canister 130 to outlet 116, has primed the outlet with vacuum by first opening then closing the canister isolation valve 132, so that a leak check and a flow check can be performed, with LED feedback during both leak checking and flow checking steps.
4. In response to detecting seven or more button presses in rapid succession, the system 100 begins sampling and presents a fourth visual indication to indicate the beginning of sampling, such as a green LED for two minutes. During sampling, the system 100 outputs a fifth visual indication, such as a green LED flash every 15 seconds to indicate sampling is ongoing.
5. In response to detecting a sustained button press (e.g., held for 5 seconds), the system 100 stops sampling and clears any existing error messages.

In some embodiments, a variation of method 400 can be performed to enable continuous monitoring at a respective location. That is, by visiting the site once every 1-2 weeks just as sampling finishes, another canister 130 can be attached to start the next 1-2 week collection. In this way, continuous monitoring of the air at that location can be performed, as this is the most certain way to determine whether the several hundred or more compounds compatible with this technique are at safe levels or not.

Continuous monitoring is similar to discrete monitoring events, except somewhat easier, as the sampling will occur the same way every time, so re-programming isn't necessary. Continuous monitoring begins with the performance of the operations described above with reference to one or more (or all) of 402 through 412. At a predetermined time, such as at the time the current canister 130 is expected to be filled to the desired level, a clean replacement canister at vacuum is brought to the sampling site.

Removing the canister 130 includes closing the canister before disconnecting it from the sampling system 100 to avoid introducing additional air to the canister 130 during this process. Removing the canister 130 from the sampling system 100 will cause the sampling system 100 to register a rapid increase in pressure due to transitioning from measuring the negative pressure of the canister to measuring the local atmospheric pressure. In response to detecting this increase in pressure, the sampling system 100 determines that the canister 130 has been disconnected. The sampling data (e.g., pressure data corresponding to plot 300 and/or valve pulse time corresponding to plot 350) can be collected as described above with reference to 416. Retrieving the sampling data optionally occurs while the canister 130 is connected. Pressure data includes the final pressure in the canister 130 at the end of the sampling process and at the time of sampling system 100 and/or canister 130 retrieval from the sampling location to verify no additional sample was collected after the end of the sampling process.

Once the canister 130 containing the collected sample is removed, a clean, evacuated canister 130 can be attached in its place and sampling can be started as described above with reference to 412. The new canister 130 can be left in place to continue collecting sample and the canister 130 containing the previously-collected sample can be analyzed according to 416.

Techniques described herein, such as the sampling system 100 and/or method 400, can provide a simple yet quantitative and sensitive technique for measuring of VOCs in indoor or outdoor air, and in particular for creating accurate long term sampling events to assess average risk factors for the compounds collected. By sampling for longer periods at a constant rate, the number of analyses needed to cover a long time period drops substantially, and therefore the cost of performing this monitoring. For example, setting up a real time GCMS analyzer with a front end preconcentrator to reach low part per trillion levels can cost $200,000 per site, not to mention the time and cost for technical operators to run them, and the cost of maintenance. Instead, continuously collecting the air at a given location can be done using this invention with an initial investment of less than 1% of this amount, allowing hundreds of times the number of areas to be monitored for the same investment. This long-term sampling solution allows the determination of accurate time weighted averaged concentrations for many compounds that create risk factors for the general population due to their carcinogenic nature, but also for pregnant women and for children during their first several years of life. Many chemicals found in indoor air are endocrine disrupters that can affect fetal and adolescent development, potentially causing Autism and other disorders that have been on the rise over the past several decades, possibly due to the increased level of these endocrine disrupters in the environment. Many researchers believe that exposure to chemicals in food, air, water, and clothing are the reason for these developmental issues, and an improved device for monitoring Indoor Air Quality over extended periods could be used in combination with epidemiological studies to ascertain which chemicals are most likely responsible for these and other disorders.

The new long term vacuum sampler can be used in other air quality determinations, such as monitoring air in hospitals, office buildings, inside of vehicles, in schools, and other locations. Submarine air can also be ideally monitored using this technology to create a history of contamination levels onboard these vessels, as can air in low or zero gravity locations such as on space stations. Cabin air quality on commercial and military aircraft can also be monitored by selecting a sampling time that covers the duration of the flight, and in this case the system can be set to sample only up to whatever cabin pressure altitude will be used on that aircraft.

Some examples of the disclosure are directed to a sampling system, comprising: a filtered inlet; a first valve coupled to the filtered inlet; an outlet configured to fluidly couple to a canister; and a pressure sensor fluidly coupled to the outlet and configured to measure a pressure inside the canister, wherein the sampling system is configured to perform a method comprising: in response to one or more criteria for beginning sampling being met, beginning a sampling process, the sampling process including: pulsing the first valve open for variable on times, wherein the variable on times change throughout the sampling process and, during each variable on time, a predetermined fraction of a volume of the canister is filled with sample. Additionally or alternatively, in some examples filling the predetermined fraction of the volume of the canister with sample during each variable on time thereby maintains a substantially constant fill rate of the canister during a duration of the sampling process. Additionally or alternatively, in some examples the method further includes: at a respective time, measuring a first pressure inside the canister and pulsing the first valve open for a first variable on time in accordance with the first pressure; and at a different time from the respective time, measuring a second pressure inside the canister and pulsing the first valve open for a different variable on time, wherein the different variable on time is greater than the first variable on time in accordance with a determination that the second pressure is greater than the first pressure. Additionally or alternatively, in some examples the sampling system further includes a temperature sensor configured to sense a temperature of the canister, wherein the sampling process includes: measuring a first temperature of the canister at a first time during the sampling process and adjusting a measurement of the first pressure in accordance with the first temperature; and measuring a second temperature of the canister at a second time during the sampling process and adjusting a measurement of the second pressure in accordance with the second temperature. Additionally or alternatively, in some examples the method further includes: monitoring a pressure inside the canister; and in accordance with a determination that the pressure inside the canister reaches a predetermined pressure level, ceasing pulsing the first valve open. Additionally or alternatively, in some examples the sampling process includes increasing the variable on times during which the first valve is pulsed open while the predetermined fraction of a volume of the canister that is filled with sample during each variable on time remains constant over time. Additionally or alternatively, in some examples the one or more criteria for beginning sampling include one or more of a predetermined time of day being reached, detecting an input received by a switch included in the sampling system, and/or receiving an indication to begin sampling from an electronic device. Additionally or alternatively, in some examples the method further comprises: performing the sampling process while a first canister is coupled to the sampling system; after performing the sampling process while the first canister is coupled to the sampling system, sealing the first canister with a second valve; after sealing the first canister with the second valve, de-coupling the first canister from the sampling system; and repeating the sampling process while a second canister is coupled to the sampling system.

Some examples of the disclosure are directed to a method comprising: at a sampling system including a filtered inlet, a first valve coupled to the filtered inlet, an outlet configured to fluidly couple to a canister, and a pressure sensor fluidly coupled to the outlet and configured to measure a pressure inside the canister: in response to one or more criteria for beginning sampling being met, beginning a sampling process, the sampling process including: pulsing the first valve open for variable on times, wherein the variable on times change throughout the sampling process and, during each variable on time, a predetermined fraction of a volume of the canister is filled with sample. Additionally or alternatively, in some examples filling the predetermined fraction of the volume of the canister with sample during each variable on time thereby maintains a substantially constant fill rate of the canister during a duration of the sampling process. Additionally or alternatively, in some examples the method further includes at a respective time, measuring a first pressure inside the canister and pulsing the first valve open for a first variable on time in accordance with the first pressure; and at a different time from the respective time, measuring a second pressure inside the canister and pulsing the first valve open for a different variable on time, wherein the different variable on time is greater than the first variable on time in accordance with a determination that the second pressure is greater than the first pressure. Additionally or alternatively, in some examples the sampling system further includes a temperature sensor configured to sense a temperature of the canister, and the sampling process further includes: measuring a first temperature of the canister at a first time during the sampling process and adjusting a measurement of the first pressure in accordance with the first temperature; and measuring a second temperature of the canister at a second time during the sampling process and adjusting a measurement of the second pressure in accordance with the second temperature. Additionally or alternatively, in some examples the method further includes monitoring a pressure inside the canister; and in accordance with a determination that the pressure inside the canister reaches a predetermined pressure level, ceasing pulsing the first valve open. Additionally or alternatively, in some examples the sampling process includes increasing the variable on times during which the first valve is pulsed open while the predetermined fraction of a volume of the canister that is filled with sample during each variable on time remains constant over time. Additionally or alternatively, in some examples the one or more criteria for beginning sampling include one or more of a predetermined time of day being reached, detecting an input received by a switch included in the sampling system, and/or receiving an indication to begin sampling from an electronic device. Additionally or alternatively, in some examples the method further includes performing the sampling process while a first canister is coupled to the sampling system; after performing the sampling process while the first canister is coupled to the sampling system, sealing the first canister with a second valve; after sealing the first canister with the second valve, de-coupling the first canister from the sampling system; and repeating the sampling process while a second canister is coupled to the sampling system.

Some examples of the disclosure are directed to a non-transitory computer readable storage medium storing instructions that, when executed by one or more processors in communication with a sampling system including a filtered inlet, a first valve coupled to the filtered inlet, an outlet configured to fluidly couple to a canister, and a pressure sensor fluidly coupled to the outlet and configured to measure a pressure inside the canister, cause the sampling system to perform a method comprising: in response to one or more criteria for beginning sampling being met, beginning a sampling process, the sampling process including: pulsing the first valve open for variable on times, wherein the variable on times change throughout the sampling process and, during each variable on time, a predetermined fraction of a volume of the canister is filled with sample. Additionally or alternatively, in some examples filling the predetermined fraction of the volume of the canister with sample during each variable on time thereby maintains a substantially constant fill rate of the canister during a duration of the sampling process. Additionally or alternatively, in some examples the method further includes: at a respective time, measuring a first pressure inside the canister and pulsing the first valve open for a first variable on time in accordance with the first pressure; and at a different time from the respective time, measuring a second pressure inside the canister and pulsing the first valve open for a different variable on time, wherein the different variable on time is greater than the first variable on time in accordance with a determination that the second pressure is greater than the first pressure. Additionally or alternatively, in some examples the sampling process includes increasing the variable on times during which the first valve is pulsed open while the predetermined fraction of a volume of the canister that is filled with sample during each variable on time remains constant over time.

Some embodiments of the disclosure are directed to a sampling process that includes, at predetermined time intervals according to the instructions, opening the valve to introduce a portion of a gas sample into the canister for a respective on time; and closing the valve after the respective on time has elapsed since opening the valve. In some embodiments, the respective on time is based on introducing a predetermined mass of the portion of the gas sample. In some embodiments, the sampling process includes opening the valve for a first on time at a first time during the sampling process to introduce the predetermined mass of the sample to the canister during the first on time and opening the valve for a second on time different from the first on time at a second time during the sampling process different from the first time during the sampling process to introduce the predetermined mass of the sample to the canister during the second on time. In some examples, the sampling process continues and includes opening the valve for additional on times that vary similarly to the first on time and the second on time.

Although examples have been fully described with reference to the accompanying drawings, it is to be noted that various changes and modifications will become apparent to those skilled in the art. Such changes and modifications are to be understood as being included within the scope of examples of this disclosure as defined by the appended claims.

1. A sampling system, comprising:
a filtered inlet;
a first valve coupled to the filtered inlet;
an outlet configured to fluidly couple to a canister; and
a pressure sensor fluidly coupled to the outlet and configured to measure a pressure inside the canister, wherein the sampling system is configured to perform a method comprising:
in response to one or more criteria for beginning sampling being met, beginning a sampling process, the sampling process including:
pulsing the first valve open for variable on times, wherein the variable on times change throughout the sampling process and, during each variable on time, a predetermined fraction of a volume of the canister is filled with sample.

2. The sampling system of claim 1, wherein filling the predetermined fraction of the volume of the canister with sample during each variable on time thereby maintains a substantially constant fill rate of the canister during a duration of the sampling process.

3. The sampling system of claim 1, wherein the method further includes:
at a respective time, measuring a first pressure inside the canister and pulsing the first valve open for a first variable on time in accordance with the first pressure; and
at a different time from the respective time, measuring a second pressure inside the canister and pulsing the first valve open for a different variable on time, wherein the different variable on time is greater than the first variable on time in accordance with a determination that the second pressure is greater than the first pressure.

4. The sampling system of claim 3, further comprising:
a temperature sensor configured to sense a temperature of the canister, wherein the sampling process includes:
measuring a first temperature of the canister at a first time during the sampling process and adjusting a measurement of the first pressure in accordance with the first temperature; and
measuring a second temperature of the canister at a second time during the sampling process and adjusting a measurement of the second pressure in accordance with the second temperature.

5. The sampling system of claim 1, wherein the method further includes:
monitoring a pressure inside the canister; and
in accordance with a determination that the pressure inside the canister reaches a predetermined pressure level, ceasing pulsing the first valve open.

6. The sampling system of claim 1, wherein the sampling process includes increasing the variable on times during which the first valve is pulsed open while the predetermined fraction of a volume of the canister that is filled with sample during each variable on time remains constant over time.

7. The sampling system of claim 1, wherein the one or more criteria for beginning sampling include one or more of a predetermined time of day being reached, detecting an input received by a switch included in the sampling system, and/or receiving an indication to begin sampling from an electronic device.

8. The sampling system of claim 1, wherein the method further comprises:
performing the sampling process while a first canister is coupled to the sampling system;
after performing the sampling process while the first canister is coupled to the sampling system, sealing the first canister with a second valve;
after sealing the first canister with the second valve, de-coupling the first canister from the sampling system; and
repeating the sampling process while a second canister is coupled to the sampling system.

9. A method comprising:
at a sampling system including a filtered inlet, a first valve coupled to the filtered inlet, an outlet configured to fluidly couple to a canister, and a pressure sensor fluidly coupled to the outlet and configured to measure a pressure inside the canister:
in response to one or more criteria for beginning sampling being met, beginning a sampling process, the sampling process including:
pulsing the first valve open for variable on times, wherein the variable on times change throughout the sampling process and, during each variable on time, a predetermined fraction of a volume of the canister is filled with sample.

10. The method of claim 9, wherein filling the predetermined fraction of the volume of the canister with sample during each variable on time thereby maintains a substantially constant fill rate of the canister during a duration of the sampling process.

11. The method of claim 9, further comprising:
at a respective time, measuring a first pressure inside the canister and pulsing the first valve open for a first variable on time in accordance with the first pressure; and
at a different time from the respective time, measuring a second pressure inside the canister and pulsing the first valve open for a different variable on time, wherein the different variable on time is greater than the first variable on time in accordance with a determination that the second pressure is greater than the first pressure.

12. The method of claim 11, wherein the sampling system further includes a temperature sensor configured to sense a temperature of the canister, and the sampling process further includes:
measuring a first temperature of the canister at a first time during the sampling process and adjusting a measurement of the first pressure in accordance with the first temperature; and
measuring a second temperature of the canister at a second time during the sampling process and adjusting a measurement of the second pressure in accordance with the second temperature.

13. The method of claim 11, further comprising:
monitoring a pressure inside the canister; and
in accordance with a determination that the pressure inside the canister reaches a predetermined pressure level, ceasing pulsing the first valve open.

14. The method of claim 9, wherein the sampling process includes increasing the variable on times during which the first valve is pulsed open while the predetermined fraction of a volume of the canister that is filled with sample during each variable on time remains constant over time.

15. The method of claim 9, wherein the one or more criteria for beginning sampling include one or more of a predetermined time of day being reached, detecting an input received by a switch included in the sampling system, and/or receiving an indication to begin sampling from an electronic device.

16. The method of claim 9, further comprising:
performing the sampling process while a first canister is coupled to the sampling system;
after performing the sampling process while the first canister is coupled to the sampling system, sealing the first canister with a second valve;
after sealing the first canister with the second valve, de-coupling the first canister from the sampling system; and
repeating the sampling process while a second canister is coupled to the sampling system.

17. A non-transitory computer readable storage medium storing instructions that, when executed by one or more processors in communication with a sampling system including a filtered inlet, a first valve coupled to the filtered inlet, an outlet configured to fluidly couple to a canister, and a pressure sensor fluidly coupled to the outlet and configured to measure a pressure inside the canister, cause the sampling system to perform a method comprising:
in response to one or more criteria for beginning sampling being met, beginning a sampling process, the sampling process including:
pulsing the first valve open for variable on times, wherein the variable on times change throughout the sampling process and, during each variable on time, a predetermined fraction of a volume of the canister is filled with sample.

18. The non-transitory computer readable storage medium of claim 17, wherein filling the predetermined fraction of the volume of the canister with sample during each variable on time thereby maintains a substantially constant fill rate of the canister during a duration of the sampling process.

19. The non-transitory computer readable storage medium of claim 17, wherein the method further includes:
at a respective time, measuring a first pressure inside the canister and pulsing the first valve open for a first variable on time in accordance with the first pressure; and
at a different time from the respective time, measuring a second pressure inside the canister and pulsing the first valve open for a different variable on time, wherein the different variable on time is greater than the first variable on time in accordance with a determination that the second pressure is greater than the first pressure.

20. The non-transitory computer readable storage medium of claim 17, wherein the sampling process includes increasing the variable on times during which the first valve is pulsed open while the predetermined fraction of a volume of the canister that is filled with sample during each variable on time remains constant over time.
